(19) United States
(12) Patent Application Publication
(10) Pub. No.: US 2011/0288883 A1
(43) Pub. Date: Nov. 24, 2011

(54) COOPERATIVE SYSTEM AND METHOD OF FULFILLING PRESCRIPTIONS

(75) Inventor: Norman D. Knoth, Clearwater, FL (US)

(73) Assignee: QEM, INC., Largo, FL (US)

(21) Appl. No.: 12/782,766

(22) Filed: May 19, 2010

(51) Int. Cl. G06Q 50/00

(52) U.S. Cl. 705/3

(57) ABSTRACT

A method of fulfilling prescription includes a plurality of cooperating pharmacies, each pharmacy having a base inventory and a cooperative inventory of medications. The base inventory is the same or substantially the same across all pharmacies while the cooperative inventory contains unique medications (only available in one cooperative inventory) or substantially unique medications (available in more than one or less than all of the cooperative inventories). When a prescription is received at an originating pharmacy, it is fulfilled from the base inventory or cooperative inventory at the originating pharmacy. If the medication of the prescription is not available in the base inventory or cooperative inventory at the originating pharmacy, then it is located in a cooperative inventory of one of the pharmacies and the prescription is transferred to that pharmacy where it is fulfilled and either provided to the customer at that pharmacy, mailed to the customer or sent to the originating pharmacy where it is picked up by the customer.

### FIELD

The present invention relates to the field of dispensing medicine and more particularly to a system and method for maintaining inventories of objects and filling requests from such inventories.

### BACKGROUND

The inventory and filling prescriptions is performed in many ways and in many locations including pharmacies, packaging plants, central locations and hospitals. In general, a customer visits a physician and the physician prescribes that the customer obtain and take or use a particular object. For the most part, the object is a medicine such as a liquid, powder, patch, pills, gelcaps, capsules, etc. The objects are not limited to such, as other non-medicine devices are sometimes prescribed.

Pharmacies or drug stores employ Pharmacists to fill prescriptions of medicines with the prescribed amount of a prescribed medicine or dose. In the past, the Pharmacist filled the prescription from a bulk package of medicine into a delivery package sized for the consumer such as a pill bottle, etc. At a time when there were relatively few prescription medicines, it was common for a local pharmacy to stock and deliver practically every possible prescription medicine. Today, several issues reduce the ability of any given pharmacy to stock substantially all medications. First, the sheer number of different medications, including brand name and generic, requires huge inventories that tie up capital, take up large amounts of space and, if not prescribed frequently, leads to waste due to expired medications. Another issue is, due to substance abuse of certain medications, local pharmacies are sometimes robbed to obtain certain medications. Another issue is related to the complexity of managing so many medications and the related possibility of delivering the wrong medication or dosage.

To overcome the issues of increasing costs associated with inventory, production, labor and the quantity of different medications, many companies that have multiple pharmacy locations have migrated to a central fill model.

Central Fill typically serves several of a company's pharmacies by consolidating certain inventories into one location and automating the prescription dispense fulfillment of as much of that inventory as possible. The number of pharmacies that a central fill location must service in order to cost-justify a reasonable return on investment varies from company to company, but most typically a very large number of pharmacies are needed for justification. This is because of very high up-front cost associated with a central fill location. Some of the normal costs associated with Central Fill are:
A building (facility)
Electrical installation for automation equipment
Tangible property taxes
Facility maintenance
Shipping costs
Computer systems
Phone systems
Automated conveyor systems
Automated prescription dispensing robots
Dispensing automation for pre-packaged medication
Ongoing service and support maintenance
Additional staffing and training costs

Although central fill has some advantages including patient safety by automating some of the fulfillment, the primary purpose is to reduce the high inventory carrying costs associated with the pharmacy environment, reduce waste due to expired medications and to increase throughput with automation. In such, the customer that requires a medication that is available only at a central fill location will experience a delay in fulfillment of their prescription. The scenario starts with the customer presenting a prescription at a local pharmacy. If the prescription is only available at the central fill location, the customer must return the next day to retrieve their medications. For this reason, certain medications that provide benefit if taken immediately are often stocked at the local pharmacy such as penicillin, amoxicillin, etc, while other medications that are often taken over long periods of time such as blood thinners, birth control pills, etc, are centrally stocked.

A typical central fill facility is usually not designed to automate and/or verify all of the medications that a company's pharmacies must dispense. By consolidating certain fast moving, volatile or costly inventories, central fill is able to automate a certain percentage of the fulfillment, for example by dispensing automation or prepackaged retrieval automation of dry-oral medication prescription such as pills. Increased patient safety for the medications that are fulfilled by automation is a natural byproduct of that automation. Central fill facilities select the medications to automate based on one or several factors. For example, high volume and/or high cost medications are chosen for the central fill location. On some occasions, it makes fiscal sense for a company to move what is known as maintenance medications to the central fill location. Maintenance medications are medications that a patient takes every month, and for which the medication type and quantity is known in advance. By moving maintenance medications to a central fill facility, a patient's monthly medications are dispensed a few days prior to their scheduled pick up or delivery, and shuttled from the central fill facility to the patient's regular pharmacy in time to meet the patient's need.

Regardless of which medication choices or unique medications that a company chooses to move to their central fill facility, it is often a much lower number of medications than the total unique medications that their pharmacies have to stock and dispense. One reason for this is the cost of automation. In some cases the fully automated robotic systems used for dispensing dry-oral medications in central fill facilities dispense around 200 different medications from cassettes (storage and delivery containers). Even if a central fill facility deploys a series of these systems together, the total number of automated medications is small compared to the larger number of medications available. The increased number of automated robotic systems increases the upfront cost as well as ongoing service and maintenance costs.

Another issue is staffing. Reducing the number of medications at the pharmacy, does not necessarily reduce the number of pharmacists required. For example, in a small, full inventory pharmacy, one pharmacist is needed. Even if the number of medications at that local pharmacy is reduced to 10% of its current value, the same pharmacist is still needed, though that pharmacist may be idle much of the time. At the same time, more staff and pharmacists are required at the central fill facility.

Historically, retail pharmacies maintain an inventory of from 1,400 to 5,000 different unique medications (depending on whether the company is an independent, grocery chain pharmacy or major drug store chain). Fully automated robots that dispense into vials and housing 200 different dry oral medications are generally cost prohibitive for such a pharmacy. Some central fill facilities utilize several of the dry-oral medication dispensing robots in order to automate a higher percentage of unique medications that their pharmacies dispense to patients. Because of the cost, less than a handful of automated dispensing machines are employed. Furthermore, each dispensing machine must work in tandem with the transfer or delivery systems which transfer the filled vials to capping stations or other automated conveyor systems for further processing before delivery to the requesting pharmacy and/or customer.

In one example, a central fill purchased and employs two prescription-dispensing machines instead of just one, housing 200 unique dry-oral medications each or a total of 400 different dry-oral medications being automated at the central fill location (assuming there are no “large moving medication” that occupy more than one potential fill location in the automation system). Assuming a grocery chain pharmacy maintaining 1,400 different medications per location, this central fill only supplants the dispensing of about 40% of the company's unique medications. Usually, 70% to 80% of a typical pharmacy's medications are dry-oral medications. In the above example, between 900 & 1,100 of the 1,400 medications used by their pharmacies would be dry-oral meds. In this, the central fill, automating 400 of those dry oral meds accounts for less than half of the unique medications the pharmacies must stock for dispensing to their patients. However, as in this example, those medications will usually account for a much higher percentage of daily dispenses than unique medications they cover, hence the cost savings for companies with enough pharmacies often cost justify a Central Fill.

However, because of the high and varied costs associated with a central fill facility, a company must have a large number of different pharmacy locations in order to cost justify one central fill location. For the right number of pharmacies within certain proximity of each other and the central fill location, central fill offers a viable cost savings based on volume.

The method of doing business that we are proposing is unique in that it has virtually none of those additional costs associated with Central Fill—yet can achieve similar cost saving results. This proposed method of doing business also provides a company's pharmacies with the ability to automate a substantially higher number of medications than is typically automated with a Central Fill facility. This proposed method of doing business incorporates a method to automate almost all of the dry oral medications, as well as cross check and verify every prescription dispensed to patients. This unique method of doing business could provide nearly complete prescription patient safety when it is properly followed and adhered to.

Many studies and reports corroborate that automation has proven to bring a much higher level of verification, control and assurance over that of manual prescriptions dispenses. This implies that with a higher number of prescriptions that are automated, a higher level of patient safety will result.

What is needed is a cooperative method of fulfilling prescriptions in which multiple inventories of medications are maintained across multiple pharmacies, thereby reducing the total number of medications at each pharmacy and the total overall inventory of medications.

### SUMMARY

A cooperative method of distributing a subset of the medications across a number of individual pharmacies reduces inventory at each or most of the pharmacies, reduces total inventory, reduces waste due to expired medications and improves customer satisfaction through prompt fulfillment of the prescriptions. The method enables automation, whereby automation in any or all of a company's pharmacies provides efficiency, cost savings and improved accuracy. In such, a much higher number of prescription fulfillments are automated and/or verified leading to an increased level of patient safety.

In one embodiment, a method of fulfilling a prescription is disclosed including (a) providing a plurality of pharmacies, each having a base inventory and a cooperative inventory and (b) accepting a prescription from a customer at an originating pharmacy of the pharmacies then (c) determining if a medication prescribed by the prescription is available in either of the base inventory within the originating pharmacy or in the cooperative inventory within the originating pharmacy. (d) If the medication is available in either of the base inventory within the originating pharmacy or in the cooperative inventory within the originating pharmacy, the prescription is fulfilled at the originating pharmacy, payment is accepted from the customer and the medication is provided to the customer. (e) If the medication is not available in either of the base inventory within the originating pharmacy or in the cooperative inventory within the originating pharmacy, (f) the medication is located in the cooperating inventory of a cooperating pharmacy of the pharmacies, (g) the prescription is transferred from the originating pharmacy to the cooperating pharmacy and (h) the prescription is fulfilled from the cooperating inventory of the cooperating pharmacy.

In another embodiment, a method of fulfilling a prescription is disclosed including (a) providing a plurality of pharmacies, each pharmacy having a base inventory and a cooperative inventory and (b) providing a computer system, the computer system having a database, the database having data indicating quantities of each medication available in each of the base and cooperative inventories. (c) Access to the database is provided at each of the pharmacies. (d) A prescription from a customer is accepted at an originating pharmacy and (e) the database is used to determine if a medication prescribed by the prescription is available in either of the base inventory within the originating pharmacy or in the cooperative inventory within the originating pharmacy. (f) If the medication is available in either of the base inventory within the originating pharmacy or in the cooperative inventory within the originating pharmacy, the prescription is fulfilled at the originating pharmacy, accepting payment from the customer and providing the medication to the customer. (g) If the medication is not available in either of the base inventory within the originating pharmacy or in the cooperative inventory within the originating pharmacy, (h) the database is used to locate the medication in the cooperating inventory of a cooperating pharmacy of the pharmacies and (i) the prescription is transferred from the originating pharmacy to the cooperating pharmacy and where (j) the prescription is fulfilled from the cooperating inventory of the cooperating pharmacy

In another embodiment, a method of fulfilling a prescription is disclosed including (a) providing a plurality of pharmacies, each pharmacy having a base inventory and a cooperative inventory and (b) providing a computer system, the computer system having a database, the database having data indicating quantities of each medication available in each of the base and cooperative inventories. (c) Access to the database is provided at each of the pharmacies. (d) A prescription from a customer is accepted at an originating pharmacy and (e) the database is used to determine if a medication prescribed by the prescription is available in either of the base inventory within the originating pharmacy or in the cooperative inventory within the originating pharmacy. (f) If the medication is available in either of the base inventory within the originating pharmacy or in the cooperative inventory within the originating pharmacy, the prescription is fulfilled at the originating pharmacy, accepting payment from the customer and providing the medication to the customer. (g) If the medication is not available in either of the base inventory within the originating pharmacy or in the cooperative inventory within the originating pharmacy, (h) the database is used to locate the medication in the cooperating inventory of a cooperating pharmacy of the pharmacies and (i) the computer system is used to transfer the prescription from the originating pharmacy to the cooperating pharmacy and where (j) the prescription is fulfilled from the cooperating inventory of the cooperating pharmacy.

### BRIEF DESCRIPTION OF THE DRAWINGS

The invention can be best understood by those having ordinary skill in the art by reference to the following detailed description when considered in conjunction with the accompanying drawings in which:

FIG. 1 illustrates a plan view of the independent pharmacy of the prior art.

FIG. 2 illustrates a plan view of the central fill solution of the prior art.

FIG. 3 illustrates a plan view of the cooperative method showing a three-pharmacy network.

FIG. 4 illustrates a first flow chart of the cooperative method.

FIG. 5 illustrates a second flow chart of the cooperative method.

### DETAILED DESCRIPTION

Reference will now be made in detail to the presently preferred embodiments of the invention, examples of which are illustrated in the accompanying drawings. Throughout the following detailed description, the same reference numerals refer to the same elements in all figures. Throughout the description the term medication is used referring to any medicinal composition, device or other object, whether a prescription is required or not. Examples of medications are tablets, capsules, caplets and gel-caps, liquids, powers, patches, electro-medical devices, canes, etc. The present invention works with most any object and can be scaled to work for much larger objects as well.

As will be shown, instead of shifting work (and staff) to a central fill facility, the cooperative method balances a company's pharmacist's workloads. For example, suppose that one pharmacy only dispensed on average 50 prescriptions per day, while another of its pharmacies dispensed 250. An embodiment of this method moves a portion of the inventory of medications from each pharmacy to other cooperative pharmacies. With an optimal balance of different or similar medications, the workload is balanced and, for example, each pharmacist in the above scenario will average 150 prescriptions per day. The workload of both pharmacies is combined and is then divided equally between them. Using the cooperative method reduces the workload at the pharmacy that was dispensing 250 daily prescriptions and thereby reduces the probability of dispensing errors or adding additional labor. The pharmacy that was dispensing only 50 prescriptions daily and was grossly underutilized is now better utilized.

It should be noted that almost every pharmacy has at least one pharmacist and one technician (2 staff members present at the pharmacy most of the time). It is known that there is a point of diminishing returns within the human capability after which both concentration and accuracy greatly decline. A person that is overtaxed can lose focus and begin to make unconscious errors. In the pharmacy world, lack of attention to details can have life and death ramifications. Many forward looking companies have historical data or studies that provide empirical data to support a number of dispenses above which another staff member should be added. Using such data, many companies know that after a certain number of prescriptions manually dispensed, either automation or an additional staff member must be added in order to lessen the possibility of dispensing a wrong medication to a patient.

In a more complex example of the prior art, consider ten pharmacies. For this example, assume that the number of prescriptions above which either automation or another staff member must be added to a 2-person pharmacy is 200 and for every 100 daily dispenses above 200, another additional staff member must be added to keep manual prescription dispensing at the minimal patient safety level. In other words, once the daily prescriptions reach 201, another staff member is added, bringing that pharmacy's total staff to 3. Once the daily prescription dispenses reach 301, yet another staff member is added, bringing that pharmacy's total staff to 4, and so on. As stated earlier these numbers are hypothetical.

The ten hypothetical pharmacies operate as follows:

| | Daily | Staff |
|---|---|---|
| Pharmacy | Load | required |
| Rx #1 | 162 | 2 |
| Rx #2 | 136 | 2 |
| Rx #3 | 236 | 3 |
| Rx #4 | 325 | 4 |
| Rx #5 | 152 | 2 |
| Rx #6 | 222 | 3 |
| Rx #7 | 223 | 3 |
| Rx #8 | 156 | 2 |
| Rx #9 | 137 | 2 |
| Rx #10 | 151 | 2 |

Note that a total of 1900 daily prescriptions are filled with a total staff of 25 and there are four pharmacies requiring additional staff because of the number of prescriptions that those pharmacies are filling daily. The total number of prescriptions dispensed is 1,900, which yields an average of 190 per pharmacy. If the daily fulfillment is divided equally between the 10 pharmacies, each pharmacy then requires two staff members because the average number (190) is below the number of daily prescriptions that require additional staff. In this example, 25% staff on payroll are not necessary to meet the unbalanced patient prescription demands.

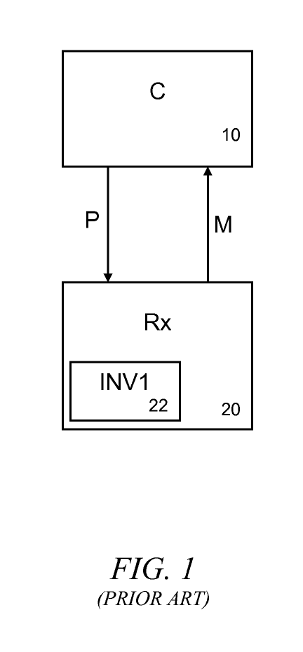

Referring to FIG. 1, a typical pharmacy 20 of the prior art has an inventory 22 of, for example, medications. Since there is no cooperative method or a central fill facility, every medication that the pharmacy 20 of the prior art dispensed comes from the inventory 22 present in that pharmacy 20. The process was simple. The customer 10 presented a prescription, P, to the pharmacy 20, the pharmacist and/or staff fulfilled the prescription, P, and provided the medication, M, to the customer 10. Payment is made and the customer 10 leaves with their medication, M. If the pharmacy 20 was out of a specific medication, then the customer 10 had to leave the pharmacy 20 and seek another pharmacy 20 that met their needs. Since the pharmacy 20 in this example has no access to a central fill facility and there is no cooperative method, the inventory 22 must contain sufficient breadth of medications to meet the needs of the typical customers 10. Historically, retail pharmacies 20 generally maintain an inventory of 1,400 to 5,000 different unique medications. This results in high inventory costs, high demands on retail space that is better utilized to present other goods to customers and waste due to expiration of certain medications, etc.

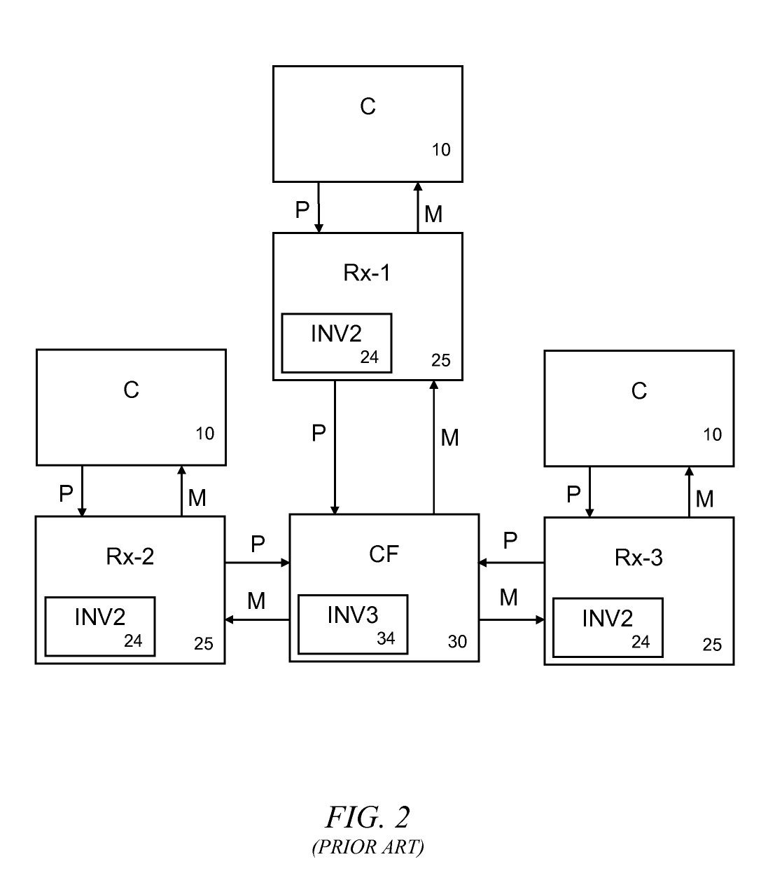

Referring now to FIG. 2, a central fill facility 30 of the prior art is shown. In the past, a central fill facility 30 was often implemented due to the high cost of automation needed at each individual pharmacy location, the high cost of inventory, waste due to expiration, etc. Recently, the cost of automation has decreased to a point where it is cost effective to provide automation at each individual pharmacy.

The example of the prior art as shown in FIG. 2 has three pharmacies 25, although the central fill method is not limited to any specific number of pharmacies 25. Each pharmacy 25 has its own local inventory 24 of medications. Often, the medications in this local inventory 24 is selected based upon frequency of prescribing, costs, safety (e.g. non-narcotic), immediate need of the customer, etc. For example, the local inventory 24 typically includes medications such as amoxicillin and penicillin but not birth control pills, sedatives, etc. Each of the pharmacies 25 has access to a central fill facility 30. The central fill facility 30 has a central fill inventory 34 of medications that are often not available in the local inventories 24 at the pharmacies 34. For example, the central fill inventory 34 typically includes medications such as birth control pills, sedatives, controlled substances, etc.

The central fill system of the prior art works as follows. The customer 10 presents a prescription, P, to one of the pharmacies 25. If the medication described in the prescription, P, is available in the local inventory 24, the medication, M, is dispensed and provided to the customer 10. If the medication described in the prescription, P, is not available in the local inventory 24, but is available in the central fill inventory 34, the prescription, P, if forwarded to the central fill facility 30 and the customer 10 leaves. The central fill facility 30 dispensed the medication, M, and sends the medication M back to the originating pharmacy 25 by courier, U.S. Mail, etc. Later, the customer 10 returns to the originating pharmacy 25 and the fulfilled medications, M, are provided to the customer 10. As stated before, there are staff, facility and maintenance costs associated with the central fill method that need be balanced with savings resulting from a reduction in overall inventory.

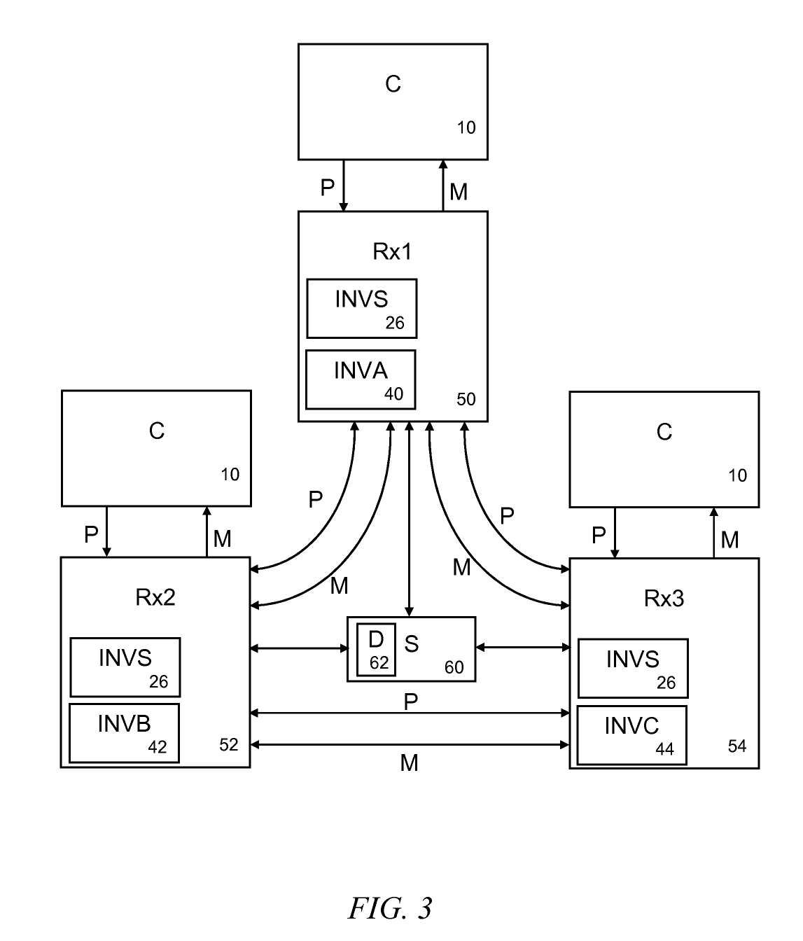

Referring now to FIG. 3, a cooperative method is described. By implementing a cooperative method of fulfilling prescriptions, the workload imbalance is reduced and staffing is minimized.

In the cooperative method, each pharmacy 50/52/54 has a base or standard inventory 26 and a cooperative inventory 40/42/44. Each pharmacy 50/52/54 has its own base inventory 26 of medications. Often, the medications in this base inventory 26 is selected based upon frequency of prescribing, costs, safety (e.g. non-narcotic), immediate need of the customer, etc. For example, the base inventory 26 typically includes medications such as amoxicillin and penicillin but not birth control pills, sedatives, etc. Each of the pharmacies 50/52/54 has a cooperative inventory 40/42/44 of medications that are often not available in the local inventories 26. For example, the cooperative inventories 40/42/44 typically include medications such as birth control pills, sedatives, controlled substances, etc.

In general, each cooperative inventory 40/42/44 includes medications that are not available in other cooperative inventories 40/42/44. In other words, it is anticipated, though not required, that each cooperative inventory 40/42/44 has a unique set of medications without duplication at different pharmacies 50/52/54. In some embodiments, a subset of medications is duplicated in several cooperative inventories 40/42/44 to, for example, expedite delivery or provide certain medications to certain regular customers 10.

In both embodiments, the cooperative fill system begins as the other methods with a customer 10 delivering a prescription, P, to one of the pharmacies 50/52/54, for example delivering the prescription, P, to the originating pharmacy 50. It is anticipated that the delivery is made in any way known, including in person, by email, facsimile, etc. If the needed medication is present in the base inventory 26 or the local cooperative inventory 40 at the originating pharmacy 50, the medications, M, are dispensed and provided directly to the customer 10. If the needed medication is not present in the base inventory 26 or the local cooperative inventory 40, it is determined if the medication is present in any one of the other cooperative inventories 42/44 at the cooperating pharmacies 52/54. If the needed medication is not present in any one of the other cooperative inventories 42/44, the prescription cannot be fulfilled by the pharmacy 50 and the customer leaves. If the needed medication is present (and in stock) in any one of the other cooperative inventories 42/44, the prescription, P, is forwarded to the cooperating pharmacy 52/54 and the cooperating pharmacy 52/54 fulfills the medications, M. Based upon the preferences of the customer 10, the customer 10 has the choice of traveling to the cooperating pharmacy 52/54 to pick up the medications, M or the customer 10 has the choice of leaving and returning to the originating pharmacy 50 when the medications arrive at the originating pharmacy 50. In the later, the medications, M, are forwarded from the cooperating pharmacy 52/54 to the originating pharmacy 50 by courier, U.S. Mail, etc.

Applying the methods of cooperative inventory to the prior example of ten pharmacies using the same assumptions and assuming an idea distribution of medications results in the following operating characteristics:

| | Daily | Staff |
|---|---|---|
| Pharmacy | Load | required |
| Rx #1 | 190 | 2 |
| Rx #2 | 190 | 2 |
| Rx #3 | 190 | 2 |
| Rx #4 | 190 | 2 |
| Rx #5 | 190 | 2 |
| Rx #6 | 190 | 2 |
| Rx #7 | 190 | 2 |
| Rx #8 | 190 | 2 |
| Rx #9 | 190 | 2 |
| Rx #10 | 190 | 2 |

The same total of 1900 daily prescriptions is now filled with a total staff reduced to 20, reducing staff by 25%.

Using our previous ten pharmacy example and using the lesser number of unique medications (1,400) the total number of medications is reduced to only about 200 to 230 in the base inventory 26 and cooperative inventory 40/42/44 of each pharmacy 50/52/54. In a sample optimal distribution, all of the pharmacies 50/52/54 have a base inventory 26 of 100 medications (those that are required quickly or are in high demand) and each pharmacy 50/52/54 has approximately 130 unique medications that are not stocked in the other pharmacies 50/52/54 in its cooperative inventory 40/42/44. Therefore, base inventories 26 of only the 100 medications in are duplicated across all pharmacies 50/52/54 and, in some embodiments, one and only one inventory of each unique medication of the combined cooperative inventory 40/42/44 is housed in one pharmacy of the cooperative pharmacies 50/52/54. Note that, for various reasons, it is anticipated that one or more medications are duplicated across any number of cooperating inventories 40/42/44. For example, two distant pharmacies 50/52/54 carry a certain medication for cases in which the customer 10 wants to drive to the fulfilling pharmacy 50/52/54 to pick up their prescription, P. In some embodiments, stocking of some of the unique medications at multiple pharmacies is also anticipated for reasons other than improving customer satisfaction such as load balancing, reducing transportation costs, increasing safety, or other reasons.

In the example of ten pharmacies 50/52/54, each pharmacy only needs enough space for approximately 230 medications instead of 1400 of the prior art. Furthermore, the cost of inventory is greatly reduced for the 1300 unique medications, the inventory of which is distributed across the ten pharmacies.

The distribution of medications is anticipated to be made using historical fulfillment data to achieve a balanced daily prescription fulfillment between all ten pharmacies. In the example, each of those ten pharmacies become the sole provider and dispenser of approximately 100 to 230 different medications and each pharmacy is responsible to fulfill all of the prescription needs for the other pharmacies for the unique medications of which it is responsible.

The cooperative method is superior to central fill, having most or similar benefits of central fill without the increased staff and facility costs. With the cooperative method, there are some increases in automation costs and distribution costs, but this is outweighed by the average inventory savings and staff savings per pharmacy.

Another benefit of the cooperative method is cross check verification of every prescription that is fulfilled by a remote pharmacy, when implemented. For any prescription medications not automated, (creams, liquids, medication patches, etc) the medication sent by the fulfilling pharmacy, in some embodiments, is verified by the ordering pharmacy. This further reduces dispensing errors such as those dispenses where the wrong dosage is given (10 mg vs. 1 mg), or the wrong medication is dispensed. There are many examples in the pharmacy world where a medication produced by a manufacturer has several different dosage strengths (such as 1 mg, 5 mg, 10 mg 50 mg, etc). Often, the actual medication looks nearly identical (except for possibly a slight tablet thickness that is often imperceptible to some people). In cases like this, it is easier to dispense the wrong medication dosage manually. The cooperative method decreases the possibility of this type of dispensing error through increased availability of automation, reduced stress on employees and added double checking.

As the number of pharmacies 50/52/54 increase, so does the complexity of knowing which medications are present in which pharmacies 50/52/54 and whether there is a sufficient supply. To handle this complexity, it is anticipated that each pharmacy have access to a computer system 60 that tracks all of the inventories 26/40/42/44 of the pharmacies 50/52/54. The overall inventory data 62 contains lists of medications available in each inventory 26/40/42/44 as well as existing quantities. By consulting the inventory data 62, the originating pharmacy 50 knows which cooperating pharmacy 52/54 can fulfill the prescription, P, if any.

An additional advantage of the cooperating method is apparent when, for example, the originating pharmacy 50 has depleted a medication in the base inventory 26. In such, the originating pharmacy 50 transfers the prescription to a cooperating pharmacy 52/54, knowing that the cooperating pharmacy 52/54 has sufficient quantity of the medication. In this example, the customer 10 travels to the cooperating pharmacy 52/54, where there prescription is typically waiting when they arrive.

The cooperative method is not restricted to companies with a large number of pharmacies. For practical purposes, a company owning ten pharmacies could achieve similar results (or better) than using a central fill system, even though they would not have nearly enough pharmacies to cost justify a central fill facility 30.

The cooperative method also decreases the number of direct store deliveries that the pharmaceutical manufacturers make to the pharmacies 50/52/54. Typically the pharmaceutical manufacturer charges a delivery charge for every delivery made to every pharmacy 50/52/54. The cooperative method reduces the number of deliveries for restocking. Before implementing the cooperative method, every pharmacy houses all of the 1,400 different medications (ten pharmacy example). Inherent with this is the possibility that for any particular medication, all ten pharmacies 50/52/54 will require restocking along with the accompanying ten delivery charges. By shifting a majority of the medications to cooperative inventories in the pharmacies 50/52/54, when restocking of a certain medication is needed, delivery is only needed to the one pharmacy 50/52/54 that has that medication in its cooperative inventory 40/42/44.

The cooperative method is not dependent on having a large number of pharmacies, does not require additional structures, services, or maintenance of a central fill location, functions when less than every pharmacy 50/52/54 that a company owns agrees to implement the cooperative method, requires no additional space beyond that which is already within each pharmacy (actually, less space), enables automation of a much larger percentage of different medications than is typically automated using a central fill facility (greater safety), provides each pharmacy 50/52/54 an opportunity to verify every prescription filled at a cooperating pharmacy 50/52/54, and achieves a greater workload balance.

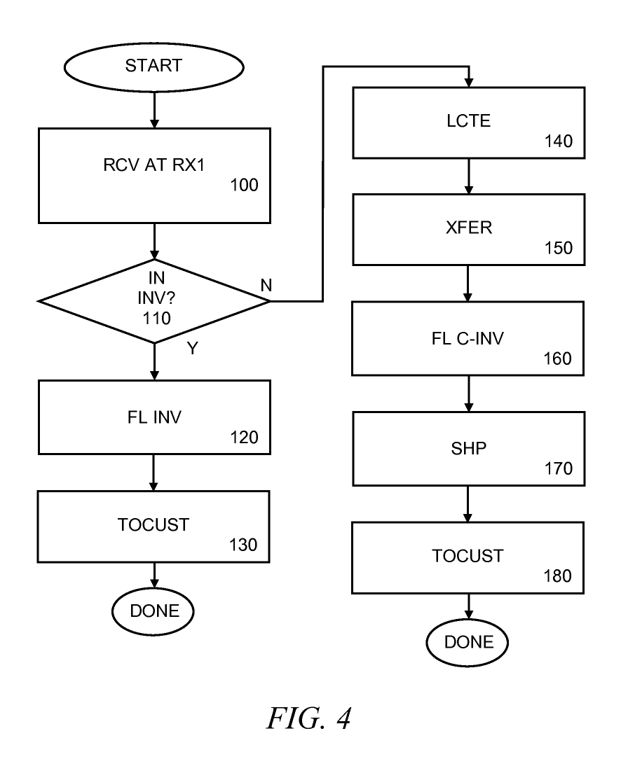

Referring now to FIG. 4, a first flow chart of the cooperative method is shown. The flow starts with receiving 100 a prescription, P, at a first pharmacy 50. The next step is to determine 110 if the medication, M, is in either the base inventory 26 or the cooperative inventory 40, the prescription, P, is fulfilled 120 at the first pharmacy 50 and provided 130 to the customer 10 upon payment. If the medication, M, is not 110 in either the base inventory 26 or the cooperative inventory 40, a cooperating pharmacy 52/54 is located 140 and the prescription, P, is transferred 150 to the cooperating pharmacy 52/54 having the medication, M, and the prescription, P, is fulfilled 160 at the cooperating pharmacy 52/54 and shipped 170 to the originating pharmacy 50 and, eventually, given 180 to the customer 10 upon payment (e.g. when the customer 10 returns to the originating pharmacy 50).

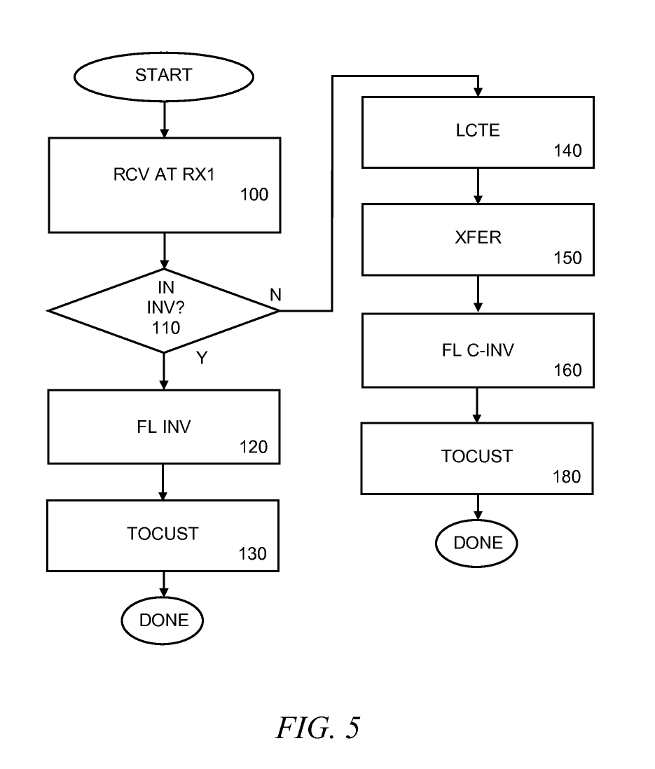

Referring now to FIG. 5, a second flow chart of the cooperative method is shown. The flow starts with receiving 100 a prescription, P, at a first pharmacy 50. The next step is to determine 110 if the medication, M, is in either the base inventory 26 or the cooperative inventory 40, the prescription, P, is fulfilled 120 at the first pharmacy 50 and provided 130 to the customer 10 upon payment. If the medication, M, is not 110 in either the base inventory 26 or the cooperative inventory 40, a cooperating pharmacy 52/54 is located 140 and the prescription, P, is transferred 150 to the pharmacy 52/54 having the medication, M, and the prescription, P, is fulfilled 160 at the cooperating pharmacy 52/54 and given or mailed 180 to the customer 10. In one scenario, the customer 10 pays for the medication, M, at the originating pharmacy 50 then travels from the originating pharmacy 50 to the cooperative pharmacy 52/54 to pick up the medication, M. In another scenario, the customer 10 travels from the originating pharmacy 50 to the cooperative pharmacy 52/54 to pick up and pay for the medication, M. Any form or sequence of payment is anticipated.

Equivalent elements can be substituted for the ones set forth above such that they perform in substantially the same manner in substantially the same way for achieving substantially the same result.

It is believed that the system and method of the present invention and many of its attendant advantages will be understood by the foregoing description. It is also believed that it will be apparent that various changes may be made in the form, construction and arrangement of the components thereof without departing from the scope and spirit of the invention or without sacrificing all of its material advantages. The form herein before described being merely exemplary and explanatory embodiment thereof. It is the intention of the following claims to encompass and include such changes.

What is claimed is:

1. A method of fulfilling a prescription, the method comprising:
(a) providing a plurality of pharmacies, each pharmacy having a base inventory and a cooperative inventory;
(b) accepting a prescription from a customer at an originating pharmacy of the pharmacies;
(c) determining if a medication prescribed by the prescription is available in either of the base inventory within the originating pharmacy or in the cooperative inventory within the originating pharmacy;
(d) if the medication is available in either of the base inventory within the originating pharmacy or in the cooperative inventory within the originating pharmacy, fulfilling the prescription at the originating pharmacy, accepting payment from the customer and providing the medication to the customer;
(e) if the medication is not available in either of the base inventory within the originating pharmacy or in the cooperative inventory within the originating pharmacy:
(f) locating the medication in the cooperating inventory of a cooperating pharmacy of the pharmacies;
(g) transferring the prescription from the originating pharmacy to the cooperating pharmacy; and
(h) fulfilling the prescription from the cooperating inventory of the cooperating pharmacy.

2. The method of claim 1, further comprising, after step (h), the steps of:
(i) sending the medication from the cooperating pharmacy to the originating pharmacy; and
(j) at the originating pharmacy, accepting payment from the customer and providing the medication to the customer.

3. The method of claim 1, further comprising, after step (g), the steps of:
(ii) traveling, by the customer, from the originating pharmacy to the cooperating pharmacy; and
(jj) at the cooperating pharmacy, accepting payment from the customer and providing the medication to the customer.

4. The method of claim 1, further comprising, after step (g), the steps of:
(iii) accepting payment from the customer at the originating pharmacy; and
(jjj) mailing the medication from the cooperating pharmacy to the customer.

5. The method of claim 1, wherein each of the cooperative inventories consist of unique medications only available in one of the cooperative inventories.

6. The method of claim 1, wherein each of the cooperative inventories consist of some unique medications only available in one cooperative inventory and some non-unique medications that are available in more than one cooperative inventory.

7. The method of claim 1, wherein a computer system is used in locating the medication in the cooperating inventory of the cooperating pharmacy of the pharmacies.

8. The method of claim 1, wherein a computer system is used in transferring the prescription from the originating pharmacy to the cooperating pharmacy.

9. A method of fulfilling a prescription, the method comprising:
(a) providing a plurality of pharmacies, each pharmacy having a base inventory and a cooperative inventory;
(b) providing a computer system, the computer system having a database, the database having data indicating quantities of each medication available in each of the base and cooperative inventories;
(c) providing access to the database at each of the pharmacies;
(d) accepting a prescription from a customer at an originating pharmacy of the pharmacies;
(e) using the database to determine if a medication prescribed by the prescription is available in either of the base inventory within the originating pharmacy or in the cooperative inventory within the originating pharmacy;
(f) if the medication is available in either of the base inventory within the originating pharmacy or in the cooperative inventory within the originating pharmacy, fulfilling the prescription at the originating pharmacy, accepting payment from the customer and providing the medication to the customer;
(g) if the medication is not available in either of the base inventory within the originating pharmacy or in the cooperative inventory within the originating pharmacy:
(h) using the database to locate the medication in the cooperating inventory of a cooperating pharmacy of the pharmacies;
(i) transferring the prescription from the originating pharmacy to the cooperating pharmacy; and
(j) fulfilling the prescription from the cooperating inventory of the cooperating pharmacy.

10. The method of claim 9, further comprising, after step (j), the steps of:
(k) sending the medication from the cooperating pharmacy to the originating pharmacy; and
(l) at the originating pharmacy, accepting payment from the customer and providing the medication to the customer.

11. The method of claim 9, further comprising, after step (j), the steps of:
(kk) traveling, by the customer, from the originating pharmacy to the cooperating pharmacy; and
(ll) at the cooperating pharmacy, accepting payment from the customer and providing the medication to the customer.

12. The method of claim 9, further comprising, after step (j), the steps of:
(kkk) accepting payment from the customer at the originating pharmacy; and
(lll) mailing the medication from the cooperating pharmacy to the customer.

13. The method of claim 9, wherein each of the cooperative inventories consist of unique medications only available in one of the cooperative inventories.

14. The method of claim 9, wherein each of the cooperative inventories consist of some unique medications only available in one cooperative inventory and some non-unique medications that are available in more than one cooperative inventory.

15. The method of claim 9, wherein the computer system is used in transferring the prescription from the originating pharmacy to the cooperating pharmacy.

16. A method of fulfilling a prescription, the method comprising:
(a) providing a plurality of pharmacies, each pharmacy having a base inventory and a cooperative inventory;
(b) providing a computer system, the computer system having a database, the database having data indicating quantities of each medication available in each of the base and cooperative inventories;
(c) providing access to the database at each of the pharmacies;
(d) accepting a prescription from a customer at an originating pharmacy of the pharmacies;
(e) using the database to determine if a medication prescribed by the prescription is available in either of the base inventory within the originating pharmacy or in the cooperative inventory within the originating pharmacy;
(f) if the medication is available in either of the base inventory within the originating pharmacy or in the cooperative inventory within the originating pharmacy, fulfilling the prescription at the originating pharmacy, accepting payment from the customer and providing the medication to the customer;
(g) if the medication is not available in either of the base inventory within the originating pharmacy or in the cooperative inventory within the originating pharmacy:
(h) using the database to locate the medication in the cooperating inventory of a cooperating pharmacy of the pharmacies;
(i) using the computer system to transfer the prescription from the originating pharmacy to the cooperating pharmacy; and
(j) fulfilling the prescription from the cooperating inventory of the cooperating pharmacy.

17. The method of claim 16, further comprising, after step (j), the steps of:
(k) sending the medication from the cooperating pharmacy to the originating pharmacy; and
(l) at the originating pharmacy, accepting payment from the customer and providing the medication to the customer.

18. The method of claim 16, further comprising, after step (j), the steps of:
(kk) traveling, by the customer, from the originating pharmacy to the cooperating pharmacy; and
(ll) at the cooperating pharmacy, accepting payment from the customer and providing the medication to the customer.

19. The method of claim 16, further comprising, after step (j), the steps of:
(kkk) accepting payment from the customer at the originating pharmacy; and
(lll) mailing the medication from the cooperating pharmacy to the customer.

20. The method of claim 9, wherein each of the cooperative inventories consist of unique medications only available in one of the cooperative inventories.
